(19) United States
(12) Patent Application Publication
(10) Pub. No.: US 2020/0093375 A1
(43) Pub. Date: Mar. 26, 2020

(54) SENSING SYSTEM AND SENSING ASSEMBLY

(71) Applicants: Chun-Hao Tseng, Taipei City (TW); Ho-Yi Chang, New Taipei City (TW)

(72) Inventors: Chun-Hao Tseng, Taipei City (TW); Ho-Yi Chang, New Taipei City (TW)

(73) Assignee: iWEECARE Co., Ltd., Taipei City (TW)

(21) Appl. No.: 16/522,673

(22) Filed: Jul. 26, 2019

Related U.S. Application Data
(60) Provisional application No. 62/736,426, filed on Sep. 25, 2018.
(60) Provisional application No. 62/736,418, filed on Sep. 25, 2018.

(51) Int. Cl. A61B 5/01; A61B 5/00

(52) U.S. Cl. CPC A61B 5/015; A61B 5/0008; A61B 5/6833; A61B 5/6813; A61B 5/6804

(57) ABSTRACT

A sensing system comprising a sensing assembly and a reader device is provided according to an embodiment of the disclosure. The sensing assembly comprises at least one sensing device and a plurality of sensors coupled to the at least one sensing device. The reader device is coupled to the sensing assembly. The sensing assembly is configured to sense a plurality of body temperatures of a user by the plurality of sensors, and the sensing assembly is further configured to transmit sensing data reflecting the sensed body temperatures to the reader device via a communication interface of the at least one sensing device.

### CROSS-REFERENCE TO RELATED APPLICATION

This application claims the priority benefits of U.S. provisional application Ser. No. 62/736,426, filed on Sep. 25, 2018 and U.S. provisional application Ser. No. 62/736,418, filed on Sep. 25, 2018. The entirety of each of the above-mentioned patent applications is hereby incorporated by reference herein and made a part of this specification.

### BACKGROUND

### Technical Field

The disclosure is related to sensing technology for sensing physical condition of a user, in particular to a sensing system and a sensing assembly.

### Description of Related Art

Generally, the physical condition (e.g., body temperature) of a user can be used to evaluate a body status of this user. For example, a basal body temperature can accurately reflect the body status of a user in many aspects. However, it is not easy for a general user to measure his basal body temperature by using a simple thermometer. Furthermore, in some cases, the body temperature measured at one single point of a human body is not enough for evaluating a body status of this human body.

### SUMMARY

A sensing system is provided according to an embodiment of the disclosure, which is capable of monitoring a plurality of body temperatures of a user by a plurality of sensors at the same time.

A sensing assembly is provided according to an embodiment of the disclosure, which is capable of monitoring a physical condition (e.g., a basal body temperature) of a user by attaching the sensing assembly to an inguinal area of the user.

A sensing system comprising a sensing assembly and a reader device is provided according to an embodiment of the disclosure. The sensing assembly comprises at least one sensing device and a plurality of sensors coupled to the at least one sensing device. The reader device is coupled to the sensing assembly. The sensing assembly is configured to sense a plurality of body temperatures of a user by the plurality of sensors, and the sensing assembly is further configured to transmit sensing data reflecting the sensed body temperatures to the reader device via a communication interface of the at least one sensing device.

A sensing assembly including a device body and an adhesive is provided according to an embodiment of the disclosure. The device body comprises a physical condition sensor. The adhesive is disposed on the device body. The sensing assembly is configured to be attached to an inguinal area of a user by the adhesive and track a physical condition of the user by the physical condition sensor.

On the basis above, in some embodiments, the sensing assembly may sense a plurality of body temperatures of a user by a plurality of sensors at the same time, and the sensing data may be transmitted to the read device for further analysis. Furthermore, in some embodiments, the sensing assembly is configured to be attached to an inguinal area of a user by the adhesive and track a physical condition of the user by the physical condition sensor, so that the physical condition (e.g., a basal body temperature) may be simply and accurately measured by a general user.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram illustrating a sensing system according to an embodiment of the disclosure.

FIG. 2 is a schematic diagram illustrating a sensing system according to an embodiment of the disclosure.

FIG. 3 is a schematic diagram illustrating multiple locations for sensing physical conditions according to an embodiment of the disclosure.

FIG. 4 is a schematic diagram illustrating a sensing assembly according to an embodiment of the disclosure.

FIG. 5 is a schematic diagram illustrating an attaching of a sensing assembly on an inguinal area of a user according to an embodiment of the disclosure.

FIG. 6 is a schematic diagram illustrating a sensing assembly with a wearable accessory according to an embodiment of the disclosure.

FIG. 7 is a schematic diagram illustrating a sensing assembly with a binding component according to an embodiment of the disclosure.

FIG. 8 is a schematic diagram illustrating a sensing assembly with an underwear according to an embodiment of the disclosure.

### DESCRIPTION OF THE EMBODIMENTS

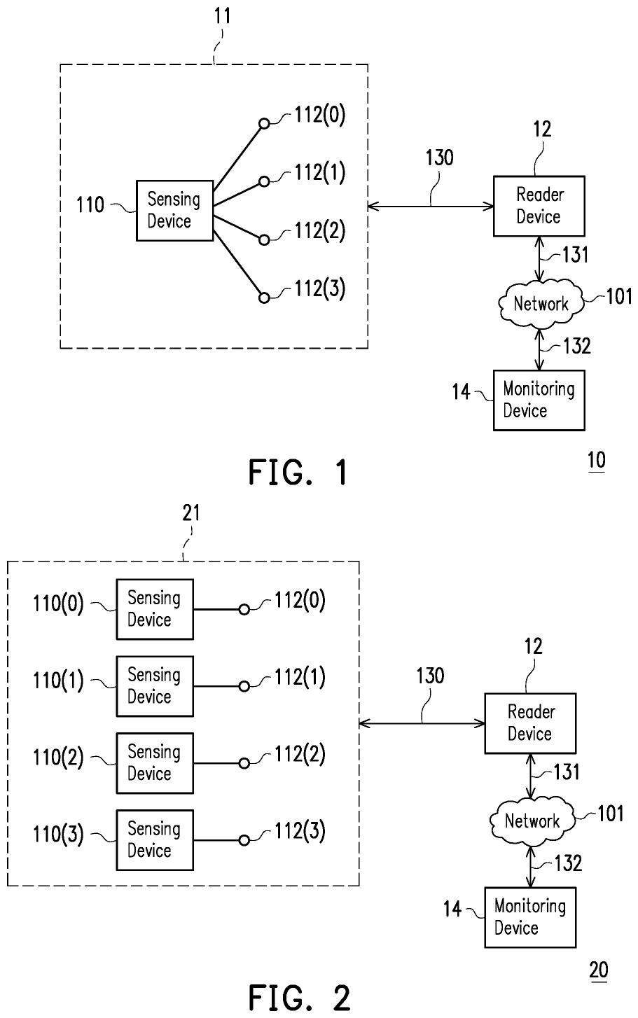

FIG. 1 is a schematic diagram illustrating a sensing system according to an embodiment of the disclosure. With reference to FIG. 1, the sensing system 10 includes a sensing assembly 11 and a reader device 12. The sensing assembly 11 includes a sensing device 110 and a plurality of sensors 112(0)-112(3) wired or wirelessly connected to the sensing device 110. It is noted that, the total number of the sensors 112(0)-112(3) may be more or less, which is not limited by the disclosure. Each of the sensors 112(0)-112(3) is configured to sense a physical condition of a user. For example, the physical condition includes at least one of pulse, heart rate, body temperature, perspiration, respiration, skin humidity, etc., but the present disclosure is not limited thereto. In the following embodiments, the body temperature is taken as an example of the physical condition.

Each of the sensors 112(0)-112(3) may generate sensing data corresponding to the sensed physical condition (e.g., the body temperature of the user). The sensing device 110 may receive the sensing data from the sensors 112(0)-112(3) and transmit the sensing data to the reader device 12 via a connection 130. For example, the sensing device 110 may include a communication interface (e.g., a wired and/or wireless communication circuit) for wired or wirelessly communicating with the reader device 12 via the connection 130.

In some embodiments, the reader device 12 may be a smart phone, a tablet computer, a notebook computer, a desktop computer, or other computing device which provides functions of data transmission, data processing and data display. For example, the reader device 12 may include a processor (e.g., CPU), a communication interface (e.g., a wired and/or wireless communication circuit), a storage device (e.g., SRAM, DRAM, ROM, HDD and/or SSD) and a display. The reader device 12 may receive the sensing data from the sensing assembly 11 (or the sensing device 110). In some embodiments, the reader device 12 may display information related to the received sensing data, such as the sensed body temperature.

In some embodiments, the sensing system 10 further includes a monitoring device 14. The monitoring device 14 may be a smart phone, a tablet computer, a notebook computer, a desktop computer, or other computing device which provides functions of data transmission, data processing and data display. For example, the monitoring device 14 may include a processor (e.g., CPU), a communication interface (e.g., a wired or wireless communication circuit), a storage device (e.g., SRAM, DRAM, ROM, HDD and/or SSD) and a display. The monitoring device 14 may receive the sensing data from the reader device 12 and display information related to the sensing data, such as the sensed body temperature. The user may monitor the physical condition (e.g., the body temperature) sensed by the sensing assembly 11 through the reader device 12 or the monitoring device 14.

In some embodiments, the reader device 12 may be referred to as a repeater or a transponder, such as a gateway, an access point, a modem, a base station or a server. The reader device 12 may transmit the sensing data to the monitoring device 14 via a network 101 (e.g., Internet). For example, the reader device 12 may decode the received sensing data and generate packets according to the decoded sensing data. Then, the reader device 12 may transmit the packets to the monitoring device 14. The monitoring device 14 may analyze the packets to obtain the information related to the sensing data, such as the sensed body temperature. The network 101 may include a public network, a private network and/or a local network, which is not limited by the disclosure.

In some embodiments, the reader device 12 may be connected to the network 101 via a connection 131, and the monitoring device 14 may be connected to the network 101 via a connection 132. In some embodiments, the reader device 12 may be connected to the monitoring device 14 directly via a peer-to-peer (P2P) connection. Furthermore, the total numbers of the reader device 12 and the monitoring device 14 may be one or more, respectively.

In some embodiments, at least one of the connections 130-132 may be compatible with one or more wireless communication protocols, such as Wifi, Bluetooth, 3G, 4G, 5G, ZigBee and so on. In some embodiments, at least one of the connections 130-132 may be compatible with a wired communication protocol, such as Ethernet.

In some embodiments, the sensing device 110 may be paired with the reader device 12 based on the Wifi, Bluetooth or other wireless communication protocols. After the pairing, the sensing device 110 and the reader device 12 can identify each other and the connection 130 can be established. In some embodiments, the reader device 12 and the monitoring device 14 may also be paired with each other for communication.

FIG. 2 is a schematic diagram illustrating a sensing system according to an embodiment of the disclosure. With reference to FIG. 2, the sensing system 20 includes a sensing assembly 21 and the reader device 12. The sensing assembly 21 includes a plurality of sensing devices 110(0)-110(3) and a plurality of sensors 112(0)-112(3) wired or wirelessly connected to the sensing devices 110(0)-110(3), respectively. Each of the sensors 112(0)-112(3) is configured to sense the physical condition (e.g., the body temperature) of a user and generate sensing data corresponding to the sensed physical condition. The sensing device 110(0)-110(3) may receive the sensing data from the sensors 112(0)-112(3), respectively, and transmit the sensing data to the reader device 12 via the connection 130. For example, each of the sensing devices 110(0)-110(3) may include a communication interface for wired or wirelessly communicating with the reader device 12 via the connection 130.

In some embodiments, the total number of the sensing devices 110(0)-110(3) is the same with the total number of the sensors 112(0)-112(3). Furthermore, the total number of the sensing devices 110(0)-110(3) (and/or the total number of the sensors 112(0)-112(3)) may be more or less, which is not limited by the disclosure.

In some embodiments, the sensing system 20 further includes the monitoring device 14. The reader device 12, the connections 130-131 and the monitoring device 14 of FIG. 2 may be the same with or similar to the reader device 12, the connections 130-131 and the monitoring device 14 of FIG. 1, respectively. In some embodiments of FIG. 1 and/or FIG. 2, the sensing system may not include the connections 131-132 and/or the monitoring device 14.

When the sensors 112(0)-112(3) are pasted on multiple body parts of a user, each of the sensors 112(0)-112(3) may sense the physical condition (e.g., the body temperature) of the user and then transmit sensing data (e.g., the temperature data) which reflects the sensed physical condition to the reader device 12 via the sensing device 110 of FIG. 1 (or the sensing devices 110(0)-110(3) of FIG. 2). Taking the sensing assembly 11 (or 21) including four sensors 112(0)-112(3) as example, the four sensors 112(0)-112(3) may be pasted on four independent and different body parts of one single user, such as four different locations of the foot of a user.

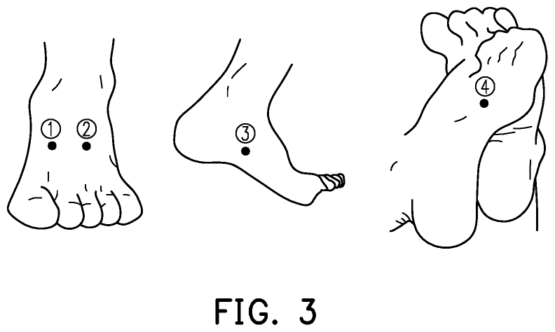

FIG. 3 is a schematic diagram illustrating multiple locations for sensing physical conditions according to an embodiment of the disclosure. With reference to FIG. 1 (or FIG. 2) and FIG. 3, the sensors 112(0)-112(3) may be pasted on the skin of the locations marked as numbers 1 to 4 of FIG. 3. As a result, the physical conditions (e.g., the body temperatures) corresponding to these four locations may be sensed by the sensors 112(0)-112(3), respectively. The sensing data reflecting the sensed physical conditions at the locations marked as numbers 1 to 4 may be transmitted to the reader device 12 via the connection 130.

It is noted that, the locations for sensing the physical conditions as shown in FIG. 3 are merely an example and not limited by the disclosure. In some embodiments, the sensors 112(0)-112(3) may be pasted on other body parts (e.g., a head, an arm, a chest, a wrist, an armpit, a groin, a forehead, a front part of nostrils or a back) of a user, which is not limited by the disclosure.

In some embodiments, the reader device 12 (or the monitoring device) may analyze the sensing data and display information related to the sensing data on a display device (e.g., a screen).

In some embodiments, the reader device 12 (or the monitoring device 14) may compare the sensing data reflecting the physical condition (e.g., the body temperature) sensed by at least one of the sensors 112(0)-112(3) with a threshold value and generate a comparison result. The reader device 12 (or the monitoring device 14) may display the comparison result on the display device. For example, the displayed information may indicate that the body temperatures sensed at locations marked as 1 to 3 of FIG. 3 are respectively higher than the threshold value and the body temperatures sensed at location marked as 4 is lower than the threshold value.

In some embodiments, the reader device 12 (or the monitoring device 14) may analyze the sensing data from at least two of the sensors 112(0)-112(3) and determine whether a threshold condition is reached according to the analysis result. Taking FIG. 3 as an example, if the analyze result reflects that there are at least half (e.g., two) or more than half of the locations having the body temperatures higher than the threshold value, the reader device 12 (or the monitoring device 14) may determine that the threshold condition is reached. However, if there is only one (or less than half) of the locations having the body temperature higher than the threshold value, the reader device 12 (or the monitoring device 14) may determine that the threshold condition is not reached.

In some embodiments, the reader device 12 (or the monitoring device 14) may generate an average physical condition (e.g., an average body temperature) according to the sensing data from at least two of the sensors 112(0)-112(3) and determine whether the threshold condition is reached according to the average physical condition. The average physical condition may reflect an average of the sensed physical conditions at multiple locations (e.g., an average of the body temperatures sensed at the locations marked as numbers 1 to 4 of FIG. 3). If the average physical condition meets a default condition (e.g., the average of the sensed body temperatures is higher than the threshold value), the reader device 12 (or the monitoring device 14) may determine that the threshold condition is reached. However, if the average physical condition does meet the default condition (e.g., the average of the sensed body temperatures is not higher than the threshold value), the reader device 12 (or the monitoring device 14) may determine that the threshold condition is not reached.

In some embodiments, the reader device 12 (or the monitoring device 14) may perform other logical operations on the sensing data from at least two of the sensors 112(0)-112(3) and display information reflect the operation result on the display device.

In some embodiments, the threshold condition being reached means that an average physical condition at multiple locations (e.g., the locations marked as numbers 1 to 4 of FIG. 3) meets a default condition, such as an average body temperature sensed at multiple locations (e.g., the locations marked as numbers 1 to 4 of FIG. 3) is higher than the threshold value. In some embodiments, once the threshold condition is reached, it reflects that an average temperature of multiple locations is higher than the threshold value. The reader device 12 (or the monitoring device 14) may display the determination result regarding whether the threshold condition being reached on the display device.

In some embodiments, the sensing device 110 of FIG. 1 (or the sensing devices 110(0)-110(3) of FIG. 2) and the sensors 112(0)-112(3) are configured to monitor the current physical condition (e.g., the current body temperature) and/or the change of the physical condition sensed at multiple locations. The reader device 12 (or the monitoring device 14) may display information related to the physical condition and/or the change of physical condition.

In some embodiments, each of the sensors 112(0)-112(3) of FIG. 2 may be a body temperature patch which may be pasted on the skin of human body. Alternatively, in some embodiments, the sensors 112(0)-112(3) of FIG. 2 may be deployed on a wearable accessory, such as a sock, a shoes, a hat, a clothes, an underwear and so on. When a user wears the wearable accessory, the sensors 112(0)-112(3) may be automatically located to multiple default locations near the human body and the temperatures at these default locations can be sensed by the sensors 112(0)-112(3).

In some embodiments, the sensing system 10 (or 20) may be used at surgery. Taking FIG. 3 as an example, before a surgery of the foot, the body temperatures at the locations marked as numbers 1 to 4 may be sensed by the sensing assembly 11 (or 21). After the surgery of the foot, the body temperatures at the locations marked as numbers 1 to 4 may be sensed by the sensing assembly 11 (or 21) again. According to the sensing and analysis result, the physical condition information (e.g., the change of the sensed body temperatures) may be displayed by the reader device 12 (or the monitoring device 14). Then, the doctor may evaluate the surgery situation according to the displayed information. For example, once the threshold condition is reached (e.g., the average temperature of multiple locations is higher than the threshold value) after the surgery, the doctor may determine that the surgery result is good. However, if the threshold condition is not reached (e.g., the average temperature of multiple locations is not higher than the threshold value) for a long time after the surgery, the doctor may determine that the surgery result is not good and execute necessary operations.

In some embodiments, the reader device 12 (or the monitoring device 14) may further provide assistance information based on the determination/analysis result. For example, the assistance information may indicate that the surgery result is good or bad and/or provide some recommendation procedures for guiding the doctor and/or the patient.

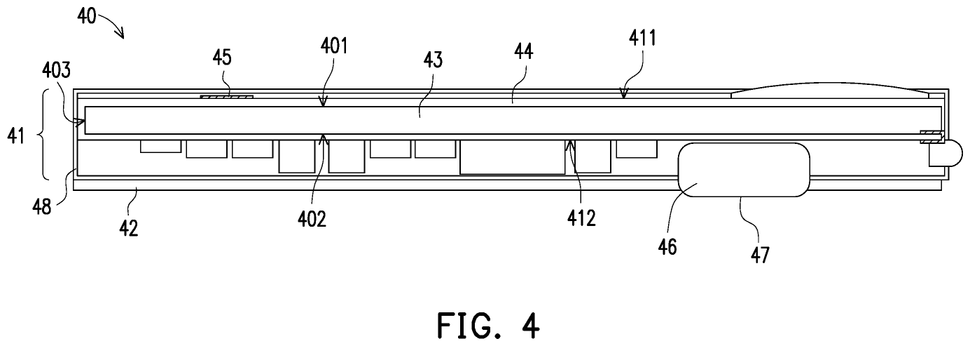

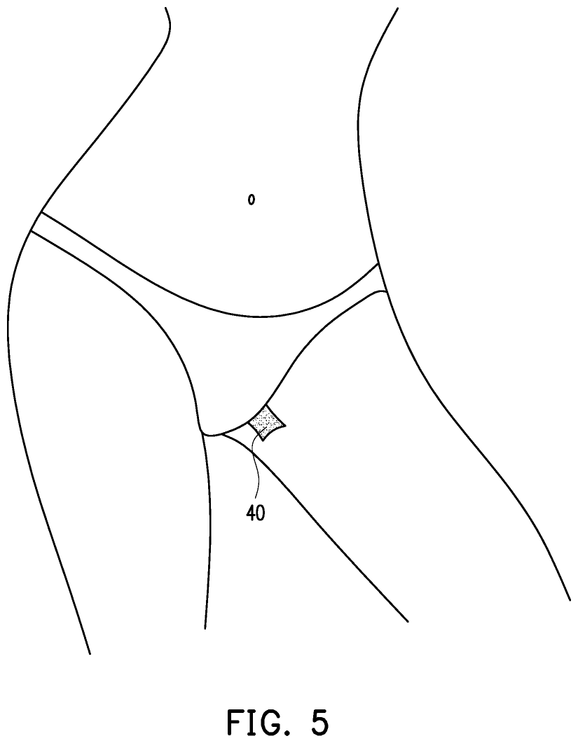

In some embodiments, to better detect physical condition of a user at an inguinal area, a sensing assembly and method for sensing physical condition of a user at an inguinal area is provided. FIG. 4 is a schematic diagram illustrating a sensing assembly according to an embodiment of the disclosure. FIG. 5 is a schematic diagram illustrating an attaching of a sensing assembly on an inguinal area of a user according to an embodiment of the disclosure.

With reference to FIG. 4, a sensing assembly 40 may be attached to a body of a user and configured to detect physical condition of the user. In some embodiments, the sensing assembly 40 includes a device body 41 and an adhesive 42. The device body 40 includes a battery unit 43, a wiring board unit 44 having a printed antenna printed 45 thereon, and a physical condition sensor 46. The adhesive 42 is disposed on the wiring board unit 44 for being attached to the body of the user. In some embodiments, the sensing assembly 40 may be attached to an inguinal area of a user to track a physical condition of the user.

In general, basal body temperature (BBT or BTP) is the lowest body temperature attained during rest (usually during sleep). It is usually estimated by a temperature measurement immediately after awakening and before any physical activity has been undertaken. This will lead to a somewhat higher value than the true basal body temperature. Accordingly, in some embodiments, the sensing assembly 40 is configured to be attached to the body of the user, so it can (continuously) monitor the basal body temperature of the user during sleep. Therefore, the basal body temperature detected by the sensing assembly 40 can better reflect the actual basal body temperature.

In addition, it is proven that inguinal site temperatures are more reflective of basal body temperature and may be less sensitive to effects of brown adipose tissue heat generation. Therefore, in some embodiments, the sensing assembly 40 is configured to be attached to the inguinal area of the user by the adhesive 42 such as a double-sided tape or the like. Generally speaking, the inguinal area means the junctional area between abdomen and thigh on either side of the pubic bone as it is shown in FIG. 5.

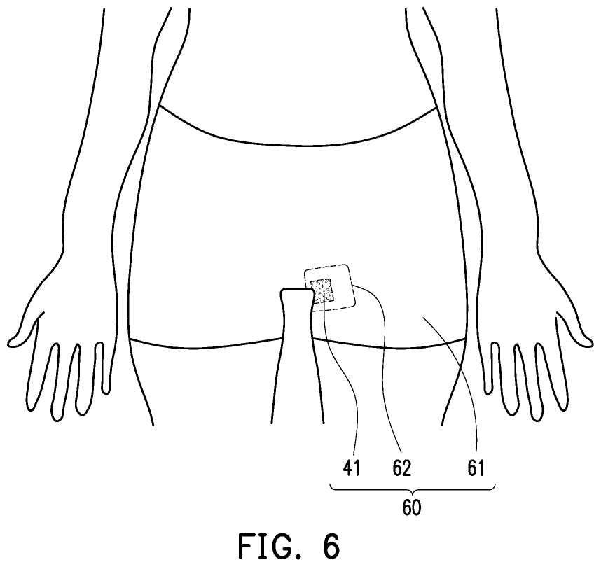

FIG. 6 is a schematic diagram illustrating a sensing assembly with a wearable accessory according to an embodiment of the disclosure. Referring to FIG. 4 and FIG. 6, a sensing assembly 60 can be a wearable accessory for the user. For example, the sensing assembly 60 may include an underwear 61, a pocket portion 62 and the device body 41. The underwear 60 can be briefs, underpants, knickers, pantie, etc. The pocket portion 62 may be sewed to an inner surface of the underwear 61, which is configured to contact the skin of the user. The location of the pocket portion 62 may be corresponding to the inguinal area of the user when the user put on the underwear 61. The device body 41, which has the same or at least similar structure of the sensing assembly 40 shown in FIG. 4, is disposed in the pocket portion 62. In some embodiments, the pocket portion 62 may have an opening for exposing a sensing region (e.g. the sensing region 47 in FIG. 4) of a physical condition sensor (e.g. the physical condition sensor 46 in FIG. 4) of the device body 41. In some embodiments, the device body 41 may be fixed to the underwear 61. For example, the device body 41 may be directly attached to the underwear 61 without being disposed in the pocket portion 62. Accordingly, the device body 41 can track physical condition (e.g., the body temperature) at the inguinal area of the user when the user put on the underwear 61.

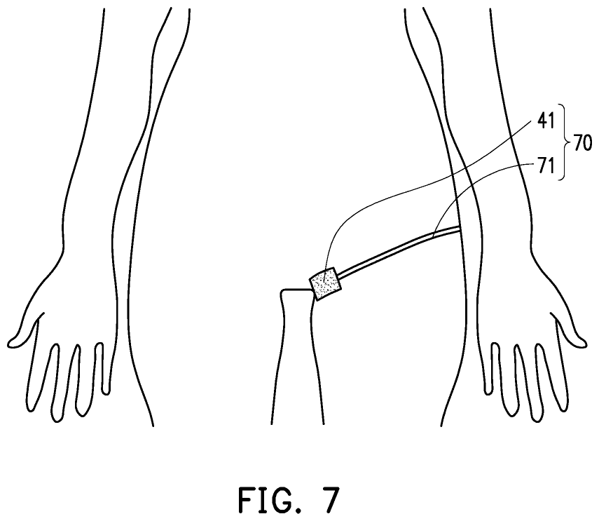

FIG. 7 is a schematic diagram illustrating a sensing assembly with a binding component according to an embodiment of the disclosure. Referring to FIG. 7, the sensing assembly 40 may include a binding component 71 and the device body 41. The device body 41 may be fixed on the binding component 71, and the binding component 71 may be an elastic band for binding on the inguinal area of the user, so that device body 41 contacts the inguinal area of the user for monitoring the basal body temperature when the binding component 71 is bound on the inguinal area of the user. In some embodiments, the binding component 71 may include an engaging component such as a buckle or the like to fix the binding component 71 onto the inguinal area of the user (e.g. highest point of the thigh). In other embodiments, the binding component 71 may be an integrally formed elastic band configured to be sleeved on the inguinal area of the user with the elasticity of the band. Other method of attaching the device body 41 to the inguinal area of the user for tracking the basal body temperature at the inguinal area may also be applied to the disclosure.

With now reference back to FIG. 1, in some embodiments, the sensing assembly 40 may also be attached to other part of the body, for example, a chest, a wrist, an armpit, a groin, a forehead, a front part of nostrils or a back of the user. The sensing assembly 40 may be configured to detect a physical condition such as pulse, heart rate, body temperature, perspiration, respiration, skin humidity, etc., but, of course, the present invention is not limited thereto.

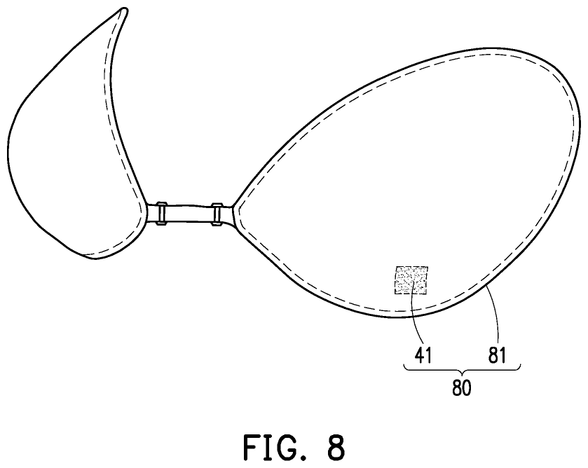

FIG. 8 is a schematic diagram illustrating a sensing assembly with an underwear according to an embodiment of the disclosure. Referring to FIG. 8, the sensing assembly 80 may include an underwear 81 and the device body 41. The underwear 81 may be a nu bra (stick-on bra), a strapless bra, a sport bra, T-shirt bra, maternity bra, or any other suitable underwear. The device body 41 may be attached to an inner surface of the underwear 81, so the device body 41 can contact the chest of the user for tracking basal body temperature at the chest of the user. Other method of attaching the device body 41 to the underwear 81 such as the pocket portion 62 of FIG. 6 described above can also be applied to the present embodiment.

In some embodiments, the sensing assembly may be a sensor chip and is configured to be implanted into a user's body for detecting physical condition of the user. For example, the sensing assembly may be implanted into an inguinal area of the user for constantly detecting basal body temperature or other physical condition of the user.

With now reference back to FIG. 1, in some embodiments, the battery unit 43 may be a lithium battery. In some embodiments, the wiring board unit 44 may be a flexible printed circuit board, and the flexible printed circuit board is bended to cover the top surface 401, the bottom surface 402 and the one of the side surfaces 403 of the battery unit 43. Therefore, owing to the flexibility thereof, the flexible printed circuit board can be bended to electrically connect two opposite surfaces, i.e., the top surface 401 and the bottom surface 402, of the battery unit 43, so that the components of the device body 41 can be disposed on the two opposite surfaces of the battery unit 43, so as to improve the space utilization of the device body 41 and further reduce the overall size of the device body 41. Also, the flexible printed circuit board (i.e., the wiring board unit 44) provides flexibility to the device body 41, so as to provide better comfort to the user when the device body 41 is in direct contact with the body of the user.

In some embodiments, the adhesive 42 may be a double-sided tape which is disposed on the second outer surface 412 of the wiring board unit 44. To be more specific, the adhesive 42 is disposed on the isolation enclosure 48 corresponding to the second outer surface 412, such that the sensing assembly 40 can be attached to the body of the user by the adhesive 42. The adhesive 42 exposes the sensing region 47 of the physical condition sensor 46, so the sensing region 47 can contact the body of the user when the sensing assembly 40 is attached to the body of the user. However, in other embodiment, the adhesive 42 may be a one-sided tape disposed on the first outer surface 411. The size of the adhesive 42 is greater than the size of the core portion, so when the adhesive 42 is disposed on the first outer surface 411, a peripheral region of the adhesive 42 would be exposed, so that the sensing assembly 40 can be attached to the body of the user by the peripheral region of the adhesive 42.

In summary, in some embodiments, the sensing assembly may sense a plurality of body temperatures of a user by a plurality of sensors at the same time, and the sensing data may be transmitted to the read device for further analysis, such as providing assistance information for surgery. Furthermore, in some embodiments, the sensing assembly is configured to be attached to an inguinal area of a user by the adhesive and track a physical condition of the user by the physical condition sensor, so that the physical condition (e.g., a basal body temperature) may be simply and accurately measured by a general user.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A sensing system, comprising:
a sensing assembly, comprising at least one sensing device and a plurality of sensors coupled to the at least one sensing device; and
a reader device, coupled to the sensing assembly,
wherein the sensing assembly is configured to sense a plurality of body temperatures of a user by the plurality of sensors, and
the sensing assembly is further configured to transmit sensing data reflecting the sensed body temperatures to the reader device via a communication interface of the at least one sensing device.

2. The sensing system of claim 1, further comprising:
a monitoring device, coupled to the reader device,
wherein the reader device is further configured to transmit the sensing data to the monitoring device, and
the monitoring device is configured to display information related to the sensing data.

3. The sensing system of claim 1, wherein the reader device is communicated with the monitoring device via a peer-to-peer connection.

4. The sensing system of claim 1, wherein a total number of the at least one sensing device is one.

5. The sensing system of claim 1, wherein a total number of the at least one sensing device is larger than one, and the plurality of sensors is connected to the at least one sensing device one to one.

6. The sensing system of claim 1, wherein the plurality of sensors are configured to be pasted on a plurality of body parts of the user to sense the body temperatures of the user at different locations.

7. The sensing system of claim 6, further comprising:
a monitoring device, coupled to the reader device,
wherein the monitoring device is configured to receive the sensing data from the reader device and analyze the sensing data, and
the monitoring device is further configured to determine whether a threshold condition is reached according to an analysis result.

8. The sensing system of claim 7, wherein an operation of determining whether the threshold condition is reached according to the analysis result comprises:
comparing the sensed body temperatures with a threshold value; and
determining whether the threshold condition is reached according to a comparison result.

9. The sensing system of claim 7, wherein an operation of determining whether the threshold condition is reached according to the analysis result comprises:
generating an average body temperature according to the sensed body temperatures; and
determining whether the threshold condition is reached according to the average body temperature.

10. The sensing system of claim 7, wherein the monitoring device is further configured to provide assistance information based on the analysis result, and the assistance information reflects a surgery result of the user.

11. A sensing assembly, comprising:
a device body, comprising a physical condition sensor; and
an adhesive, disposed on the device body,
wherein the sensing assembly is configured to be attached to an inguinal area of a user by the adhesive and track a physical condition of the user by the physical condition sensor.

12. The sensing assembly of claim 11, wherein the sensed physical condition reflects a basal body temperature of the user.

13. The sensing assembly of claim 11, further comprising:
a wearable accessory,
wherein the device body is fixed to the wearable accessory, and the device body is attached to the inguinal area of the user when the user put on the wearable accessory.

14. The sensing assembly of claim 13, wherein the device body is attached to an inner surface of the wearable accessory.

15. The sensing assembly of claim 14, wherein the wearable accessory comprises:
a pocket portion, sewed to the inner surface of the wearable accessory, and the device body is disposed in the pocket portion.

16. The sensing assembly of claim 15, wherein the pocket portion comprises:
an opening, configured to expose a sensing region of the physical condition sensor to the inguinal area of the user.

17. The sensing assembly of claim 13, wherein the wearable accessory comprises:
a binding component, configured to bind the device body to the inguinal area of the user when the user put on the wearable accessory.

18. The sensing assembly of claim 17, wherein the binding component comprises an elastic band.

19. The sensing assembly of claim 13, wherein the wearable accessory is an underwear.

20. The sensing assembly of claim 19, wherein the wearable accessory comprises one of briefs, underpants, knickers and panties.
